(19) United States
(12) Patent Application Publication
(10) Pub. No.: US 2026/0047756 A1
(43) Pub. Date: Feb. 19, 2026

(54) Low cost line-field OCT system for diabetic retinopathy

(71) Applicant: KineoLabs, Inc., Billerica, MA (US)

(72) Inventors: Jay Alan Fleischman, Greenwich, CT (US); Walid A. Atia, Jamaica Plain, MA (US)

(21) Appl. No.: 19/299,493

(22) Filed: Aug. 14, 2025

Related U.S. Application Data
(60) Provisional application No. 63/683,033, filed on Aug. 14, 2024.

(51) Int. Cl. A61B 3/10; A61B 3/00

(52) U.S. Cl. CPC A61B 3/102; A61B 3/005; A61B 3/0091

(57) ABSTRACT

A low-cost line-field parallel swept-source optical coherence tomography (OCT) system suitable for home monitoring of diabetic retinopathy and other disease states including macular degeneration is disclosed. The system includes a tunable laser coupled to an interferometer and a line-scan detector, and a single-board computer configured to control laser tuning and acquire interference data. A fixation target display is positioned to project an image to a patient's eye, and is moved in discrete steps orthogonal to the projected OCT scanline to acquire multiple B-scans at different retinal positions without mechanical beam scanning. The fixation target movement enables cross-sectional or volumetric retinal imaging using a single fixed OCT scanline, reducing system complexity and cost. The single-board computer processes line-field interferograms into depth-resolved profiles, displays images, and controls fixation target positioning. The system enables early detection and monitoring of diabetic macular edema and other retinal complications in a compact, patient-operable configuration.

### RELATED APPLICATIONS

This application claims the benefit under 35 USC 119(e) of U.S. Provisional Application No. 63/683,033, filed on Aug. 14, 2024, which is incorporated herein by reference in its entirety.

### BACKGROUND OF THE INVENTION

Optical coherence tomography (OCT) is a cross-sectional, non-invasive imaging modality that is used in many areas of medical imaging. For example, in ophthalmology, OCT has been widely used for imaging the retina, choroid and anterior segment. Functional imaging of the blood velocity and vessel microvasculature is also possible.

Fourier-domain OCT (FD-OCT) has recently attracted more attention because of its high sensitivity and imaging speed compared to time-domain OCT (TD-OCT), which uses an optical delay line for mechanical depth scanning with a relatively slow imaging speed. The spectral information discrimination in FD-OCT is accomplished either by using a dispersive spectrometer in the detection arm (spectral domain or SD-OCT) or rapidly wavelength scanning a swept laser source (swept-source OCT or SS-OCT).

Compared to SD-OCT, swept-source OCT (SS-OCT) has several advantages, including its robustness to motion artifacts and fringe washout, lower sensitivity roll-off and higher detection efficiency.

Commercial SS-OCT systems employ point scanning. At each point in the scan over the retina, the swept source is high speed swept through its wavelength scanband. Several different high-speed swept source architectures have been proposed for SS-OCT that can achieve the necessary high speed wavelength sweeping. One approach employs a semiconductor optical amplifier (SOA) based ring laser design (see for example Yun et al., “High-speed optical frequency-domain imaging” Opt. Express 11:2953 2003 and Huber et al., “Buffered Fourier domain mode locking: unidirectional swept laser sources for optical coherence tomography imaging at 370,000 lines/s,” Opt. Express 13, 3513 2005). Short cavity lasers (see for example Kuznetsov et al., “Compact Ultrafast Reflective Fabry-Perot Tunable Lasers For OCT Imaging Applications,” Proc. SPIE 7554:75541F 2010) are another example employing a MEMS tunable filter. SOA based ring laser designs have been practically limited to positive wavelength sweeps (increasing wavelength) because of the significant power loss that occurs in negative tuning.

This has been attributed to four-wave mixing (FWM) in SOAs causing a negative frequency shift in intracavity light as it propagates through the SOA (Bilenca et al., “Numerical study of wavelength-swept semiconductor ring lasers: the role of refractive-index nonlinearities in semiconductor optical amplifiers and implications for biomedical imaging applications,”Opt. Lett. 31:760-762 2006).

At the same time, other architectures exist for SS-OCT that reduce the performance requirements, i.e., wavelength sweep speed, for the swept laser sources.

Fechtig et al. in an article entitled Line-Field parallel swept source MHz OCT for structural and functional retinal imaging, Biomedical Optics Express 716, Vol. 6, No. 3, (2015) describes a system that achieves 1 MHz equivalent A-scan rates by combining a lower sweep rate laser with a linear sensor. Even earlier examples exist such as Line-Field Optical Coherence Tomography Using Frequency-Sweeping Source by Lee et al. in IEEE Journal of Selected Topics in Quantum Electronics, Vol. 14, No. 1, January 2008.

Diabetic retinopathy is a complication of diabetes that affects the eyes. It is caused by damage to the blood vessels of the retina. The disease state of diabetic retinopathy can be understood through its stages and pathological mechanisms. In its early stages, microaneurysms or small bulges in blood vessels of the retina form and often leak fluid. Over time, there is a progression of blood vessel damage with increased microaneurysms and potential blockage of some retinal blood vessels. In response, the retina will tend to grow new blood vessels for nourishment, leading to increased risk of progression. In its advanced stage, new, abnormal blood vessels (neovascularization) begin to grow in the retina and into the vitreous humor (the clear, gel-like substance that fills the eye). These new vessels are fragile and prone to bleeding, leading to potential severe vision loss or blindness. Complications include vitreous hemorrhage, tractional retinal detachment, and neovascular glaucoma. This can be treated with laser photocoagulation to reduce abnormal blood vessel growth and prevent leakage and anti-VEGF injections using medications like ranibizumab or bevacizumab to inhibit vascular endothelial growth factor and reduce neovascularization. Surgical removal of the vitreous gel is sometimes performed in cases of significant vitreous hemorrhage or retinal detachment.

OCT is essential for detecting and assessing the extent of retinal damage and edema (swelling) associated with diabetic retinopathy. One of the most critical complications of diabetic retinopathy is diabetic macular edema, where fluid accumulates in the macula, leading to vision impairment. OCT can precisely measure retinal thickness and detect fluid accumulation, enabling early diagnosis and intervention. OCT is further used to monitor the response to treatments such as anti-VEGF (vascular endothelial growth factor) injections, laser photocoagulation, and corticosteroids. By comparing OCT images before and after treatment, clinicians can evaluate the reduction in retinal thickness and fluid accumulation, determining the effectiveness of the therapy. Then, regular OCT scans allow for continuous monitoring of retinal changes over time. This is crucial for detecting any progression of diabetic retinopathy or recurrence of macular edema, enabling timely adjustments to the treatment plan. Often, OCT helps in determining the optimal timing for interventions. For example, the detection of early macular edema or neovascular changes can prompt early treatment with anti-VEGF injections, potentially preventing further progression and vision loss.

### SUMMARY OF THE INVENTION

The present invention concerns an OCT system design and method of operation that can be low cost yet capable of home use by the patient for monitoring diabetic retinopathy.

The present invention provides systems, methods, and computer-readable media for generating composite retinal images using a line-field optical coherence tomography (OCT) system without mechanically scanning the OCT beam across the retina.

In one aspect, the invention features a line-field OCT system including a swept-source OCT module configured to project a fixed OCT scanline onto a retina, a line-scan image sensor for detecting interference light from the retina along the fixed scanline, and a fixation target display positioned to project a fixation target into the field of view of an eye under examination. A system-on-module controls the fixation target display to sequentially present the fixation target at multiple positions orthogonal to the fixed OCT scanline, triggers a wavelength sweep of a tunable laser to acquire a B-scan along the scanline at each fixation target position, and compiles the acquired B-scans into a composite image of a region of the retina. By controlling the patient's gaze through movement of the displayed fixation target, the OCT scanline is effectively repositioned to different retinal locations without any mechanical translation of the OCT beam.

In another aspect, the invention features a method for generating a composite retinal image in which the patient's eye is positioned so that both the OCT scanline and the fixation target are visible. The fixation target display is controlled to present the fixation target at a first position orthogonal to the scanline, a wavelength sweep of a tunable laser is triggered, interference light is detected along the scanline to generate a first B-scan, and the B-scan is stored in memory. The fixation target is then presented at a second position orthogonal to the scanline, and the process is repeated until a predetermined number of positions has been completed. The resulting B-scans are compiled into a composite retinal image without mechanically scanning the OCT beam.

In still another aspect, a non-transitory computer-readable medium stores instructions that, when executed by a processor of a system-on-module in a line-field OCT system, cause the system to control a fixation target display to present a fixation target at a plurality of positions orthogonal to a fixed OCT scanline, trigger a wavelength sweep of a tunable laser and acquire interference data from a line-scan image sensor for each position, process the interference data into a B-scan, and compile the B-scans into a composite retinal image.

In yet another aspect, the invention features a binocular fixation OCT system in which a swept-source OCT module projects a fixed scanline into one eye of a subject, and a binocular fixation target display presents a fixation stimulus visible to the other eye. OCT interference data is acquired along the fixed scanline while the fixation stimulus is presented at different positions, and a controller combines OCT images from the different fixation stimulus positions into a composite image of the retina.

These and other embodiments provide the capability to capture multi-position retinal data using a fixed OCT scanline, reducing system complexity and cost by eliminating mechanical beam scanning, while enabling home monitoring of retinal conditions such as diabetic retinopathy.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic side view of a cat's-eye tunable laser free space coupled to an interferometer in a line-field swept source OCT system;

FIG. 2 is a top view of the interferometer according to the current embodiment;

FIG. 3 is a schematic drawing showing the movement of the fixation target relative to the projected scan line from the visual perspective of the patient; and

FIG. 4 is a flow diagram illustrating an example process for automated multi-position retinal imaging using the OCT system of the present invention, in which a system-on-module controls a fixation target display to sequentially present a fixation target at different positions to reposition the patient's gaze and thereby acquire B-scans at multiple retinal locations without mechanically scanning the OCT beam.

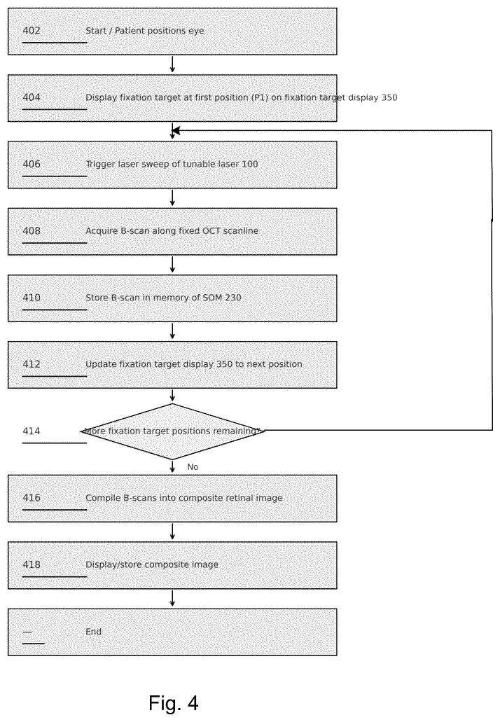

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

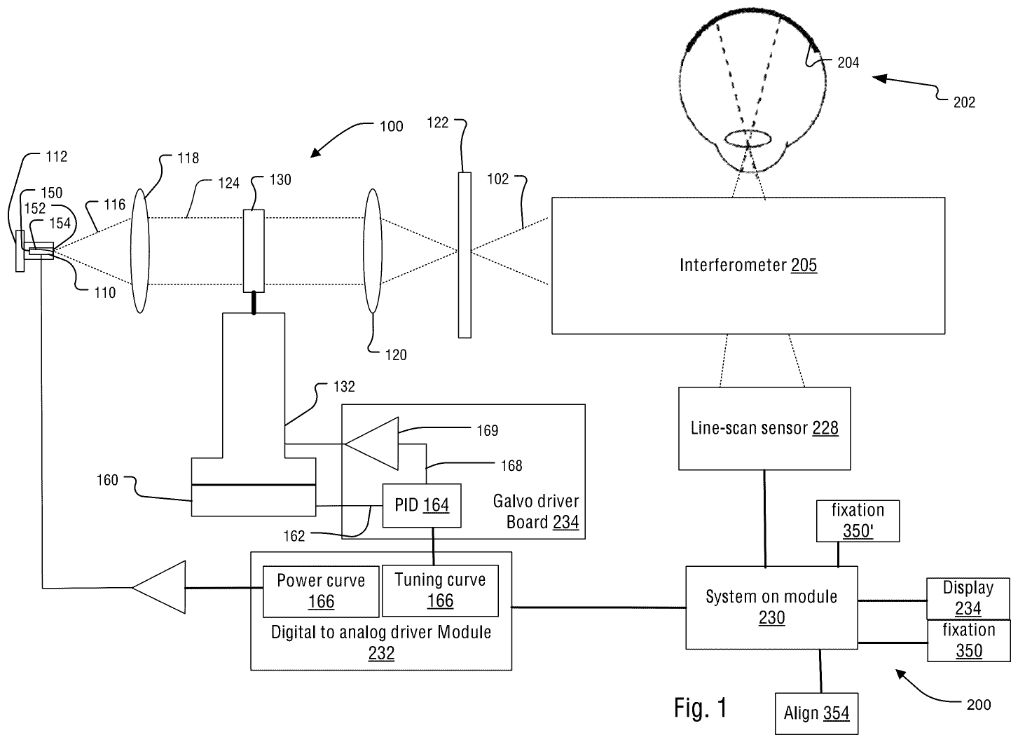

FIG. 1 shows a line-field parallel swept OCT system 200 with a cat's-eye tunable laser 100 coupled to a line-scan or line-field sensor 228 via interferometer 205.

The laser's amplification is provided by a GaAlAs gain chip 110, in one example. The gain chip 110 amplifies light in the wavelength range of about 800 to 900 nanometers. Preferably its center wavelength is around 840 nanometers, which is useful for applications such as ophthalmic imaging and other diagnostic uses because of the water window (650 to 950 nm) at these wavelengths. Another advantage of this wavelength range is that it can be detected with standard cameras with silicon-based imager chips. Specifically, the output is detected with silicon, e.g., complementary metal-oxide-semiconductor (CMOS) or charge-coupled device (CCD), imagers.

In the preferred current embodiment, the gain chip 110 is mounted in a TO-can type hermetic package 112. This protects the chip 110 from dust and the ambient environment including moisture. In some examples, the TO-can package has an integrated or a separate thermoelectric cooler 114.

The chip 110 is preferably a single angled facet (SAF) edge-emitting chip. As such, it has a high reflectivity (HR) coated rear facet 150. It has an antireflective (AR) coated front facet 152. In addition, for improved performance, it has a curved ridge waveguide 154 that is perpendicular to the rear facet 150 but is angled at the interface with the front facet 152. This angling at the front facet along with the AR coating reduces reflections at the front facet reflectivity by up to 40 dB and significantly improves laser performance by reducing parasitic reflections that can otherwise lead to non-smooth tuning and mode-hopping.

The free space beam 116 from the package 112 is diverging in both axes (x, y). It is collimated by a collimating lens 118. The resulting collimated beam 124 is received by a cat's eye focusing lens 120, which focuses the light onto a cat's eye mirror/output coupler 122. This defines the other end of the laser cavity, extending between the mirror/output coupler 122 and the back/reflective facet of the gain chip 110.

The collimated light 124 between the collimating lens 118 and the cat's eye focusing lens 120 passes through a thin film interference bandpass filter 130. This provides a pass band of approximately 0.3 nanometers (nm) full width at half maximum (FWHM) for OCT applications. More generally, its pass band is between 0.2 nm and 0.5 nm FWHM, or more generally between 0.1 nm and 2 nm FWHM. Even more generally, it is between 0.05 nm to 5 nm FWHM.

The bandpass filter is held on an arm of an angle control actuator 132 that changes the angle of the bandpass filter 130 to the collimated light 124. Generally, the angle is modulated over a range of greater than 10 degrees and typically greater than 20 degrees.

Currently, the angle is changed between 110 degrees to about 130-140 degrees or more, measured between the plane of the filter 130 and the axis of the beam 124. In one example, the angle control actuator is a galvanometer. In other examples, the angle control actuator 132 is a servomotor or an electrical motor that continuously spins the bandpass filter 130 in the collimated beam 124. This allows for tilting of the bandpass filter 130 with respect to the collimated beam 124 to thereby tilt-tune the filter and thus change the passband to scan or sweep the wavelength of the swept laser 100.

Tuning speed specifications for a galvanometer generally range from 0.1 Hz to 50 kHz. For higher speeds, a 25 kHz resonant galvanometer can be used with bi-directional tuning, but higher and lower speeds can be used. Wavelength tuning speed is usually given in nm/sec, so for a 100 Hz tuning speed ideal for retinal imaging applications where a line-speed camera at 100 kHz will give 1000 sampled bandwidth points and 70 nm tuning range, this would give 70 nm/10msec=7000 nm/sec. In general, the tuning speed should be between 3,000 nm/sec and 11,000 nm/sec or higher.

For retinal or industrial imaging with low-cost CMOS or CCD cameras, 840 nm center wavelength is an ideal water window. The tuning range is usually minimally 30 nm of tuning range. Preferably, the tuning range is closer to 60nm or 70nm or more. This provides good resolution of <8 micrometers in air. In general, the tuning range should be between 30 nm and 100 nm.

The size of the collimated beam 124 is important for many applications. As a general rule, a smaller beam results in higher divergence resulting in a larger cone half angle (CHA). This reduces the minimum line width over angle for a tunable filter. In the current embodiment, the collimated beam is preferably not less than, i.e., greater than, 1 millimeter (mm) FWHM and is preferably greater than 2 mm FWHM for retinal OCT application. It can be smaller, however, for many spectroscopy applications in the infrared, visible or ultraviolet. In general, the CHA should be less than 0.04×0.02 degrees and preferably about 0.02×0.01 degrees or less.

The light from the gain chip is polarized. In the common architectures, the polarization is horizontal or parallel to the epitaxial layers of the edge-emitting gain chip 110. In the one configuration, the filter is oriented to receive the S polarization in order to maintain narrow line width of the filter as it is tilt tuned. On the other hand, the P polarization broadens somewhat at large tilt angles. S polarization has higher loss at larger tilt angles than P. Therefore, the filter design needs to address these issues by providing a low enough loss across the tuning band for S, in the current embodiment. However, in some examples, the P polarization is used to provide higher power.

In general, the present cat's-eye configuration provides a number of advantages. It provides low loss, low tolerance, repeatable stable operation since it provides for a lower angle wavelength change over grating-based lasers.

The mirror/output coupler 122 will typically reflect about 80% of the light back into the laser's cavity and transmit about 20% of light. More generally, the mirror/output coupler can reflect from 10% to 99% of light (transmitting 90% to 1%, respectively), depending on the output power and laser cavity loss desired. Higher reflectivity results in lower loss cavities and thus wider laser tuning range where gain exceeds loss, but results in lower output power. In typical operation, the mirror/output coupler 122 reflects less than 90%.

Here, the diverging beam 102 from the mirror output coupler 122 is sent to the interferometer 205.

The angle control actuator 132 is operated as a servomechanism. In the illustrated embodiment, the angle control actuator 132 is a servo controlled galvanometer with an encoder 160. The encoder 160 produces an angle signal 162 indicating the angle of the galvanometer and thus the filter 130 to the collimated beam 124. Preferably, the encoder is an optical encoder and is often analog.

The galvanometer 132 is operated by a galvo driver board 234 that receives the angle signal 164. A PID (proportional-integral-derivative) controller 164 is implemented on the galvo driver board 234 and compares the instantaneous angle signal 162 to a desired angle dictated by a tuning curve. The PID controller 164 produces the control function 168 that is used to drive the windings of the galvanometer 132 via an amplifier 169.

In the illustrated example, the OCT system 200 is employed for ophthalmic analysis of a human eye 202 and specifically the retina 204.

Light in the form of free space beam 102 from the laser 100 passes to interferometer 205 that couples light between line-scan sensor 228 and the sample 202 such as a patient's eye.

The line field sensor typically has a linear array of at least 512 pixels, and often at least 1024 or 2048 pixels, to detect interference signals for a line. In a current example, the linear array is a few pixels wide such as between 2 and 10 pixels wide. Often the pixels can be binned in this lateral axis for higher sensitivity.

In the current implementation, the OCT system 200 is controlled by a single board computer 230. Specifically, it is System on Module (SOM) that includes a graphic processing unit (GPU), central processing unit (CPU), memory, power management, and high-speed interfaces. Currently a Jetson Orin series module is used from NVIDIA Corporation.

The SOM 230 controls a digital to analog driver module 232 which principally controls the drive to the chip 110 and the angle control actuator/galvanometer 132. In more detail, the digital to analog driver module 232 includes a tuning curve module that stores a specified tuning function for the angle of the filter 130. This is supplied to the PID controller 164, which tries to minimize the error between the angle signal 162 and the tuning curve across the wavelength sweep of the laser 100. Often, the desired tuning curve is stored in a look up table or is generated algorithmically. Often this is an approximately sawtooth or triangular waveform.

The output from the line field sensor 228 is readout by the SOM 230. The results can be stored in the SOM 230 and/or displayed on display 234. The Fourier transform of the interference light performed by the GPU within the SOM 230 at the different wavelengths or frequencies of the swept laser 100 reveals the profile of scattering intensities at different path lengths, and therefore scattering as a function of depth (z-direction) in the sample (see for example Leitgeb et al., “Ultrahigh resolution Fourier domain optical coherence tomography,” Optics Express 12(10): 2156 2004). The profile of scattering as a function of depth for a point is called an axial scan (A-scan). The combination of the projected line and line-scan sensor 228 produces a cross-sectional image (tomogram or B-scan) of the patient's eyes.

In addition, the SOM 230 further controls a fixation target display 350 and records and possibly displays images captioned by an alignment camera 354.

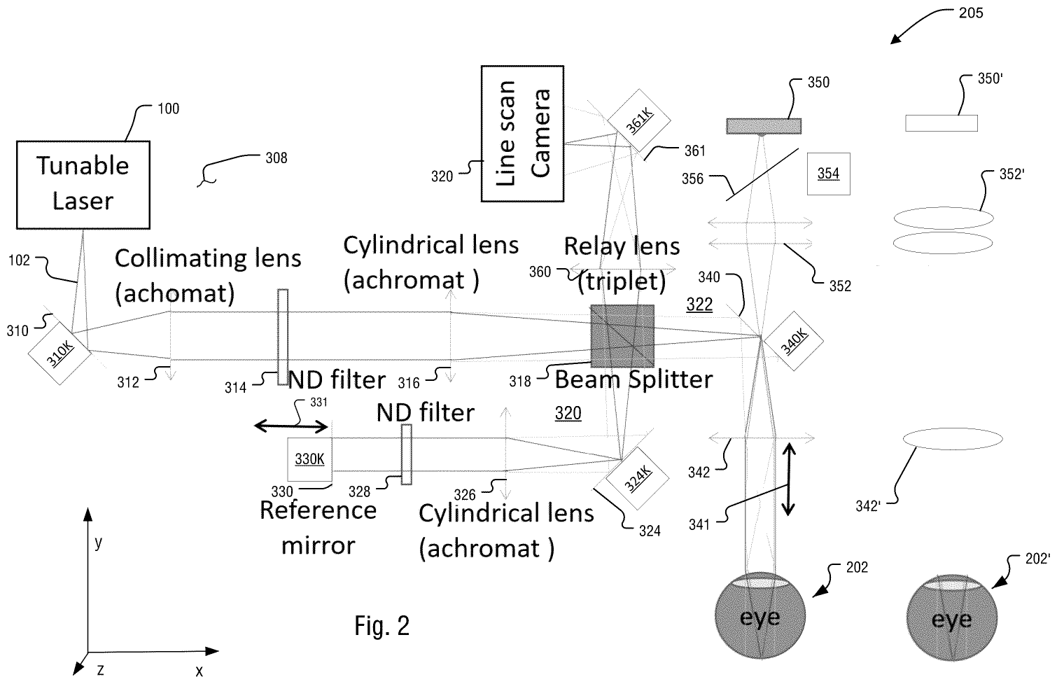

FIG. 2 shows the details of the interferometer 205 and its interfacing with the tunable laser 100 and line scan camera 320.

The free space beam 102 from the laser 100 is diverging. It is received by a mirror 310 mounted on a kinematic mount to a bench 308. The kinematic mount 310K minimally provides for adjusting the direction of the light in the x-y plane, i.e. in azimuth and elevation relative to the surface of the bench 308. In some examples, the kinematic mount 310K provides for adjusting the direction of the light in all three directions. This enables alignment of the beam for subsequent optics.

A series of components function as line-forming optics. They convert the light from the laser 100 into a line or more specifically a rectangular profile with an aspect ratio of at least 10 to 1 and typically greater than 100:1, and often 400:1, or more, measured at FWHM. That is, when looking along its optical axis, the light from the line-forming optics has a line or more specifically a high aspect ratio rectangular two-dimensional profile that is at least 10 times longer along the z-axis than along y-axis, for example, measured at the FWHM.

A collimating lens 312 of the line-forming optics collimates the beam from mirror 310. Preferably collimating lens 312 is an achromat. This achromatic lens is designed to minimize the effects of chromatic aberration across the scan band of the laser 100. Chromatic aberration is a problem that occurs when different wavelengths of light are focused at different points, resulting in a blurry image. Currently, achromatic collimating lens 312 uses two lenses made of different materials to correct for chromatic aberration over the scan band.

A neutral density filter 314 is provided to lower the power of the beam such as by lowering the power by 50% or more.

Next a cylindrical achromat lens 316 is provided to form the beam into a line. In the illustrated example, the lens focuses the light in the x-y plane so that the line extends in the direction of the z-axis.

A cube beam splitter 318 next divides the laser light between a reference arm 320 and a sample arm 322.

In the reference arm 320, a reference arm mirror 324 is mounted to the bench 308 via a kinematic mount 324K. The reference arm mirror 324 is provided to fold the beam path and also allow for alignment. Next a cylindrical achromatic lens 326 collimates the beam. A reference arm neutral density filter 328 adjusts the power of the reference arm light and a reference arm mirror 330 is mounted on a linear motion rail 331 which in turn mounted to the bench 308. The reference arm mirror 330 is moved on the linear motion rail 331 and moved by a linear motion actuator 331A to define and control the end of the reference arm and thus control the delay to path match to the sample 202. Preferably the reference arm mirror 330 is mounted to the linear motion rail 331 via a kinematic mount 330K.

In the sample arm 320, a sample arm dichroic mirror 340 is held on a kinematic mount 340K, which is mounted on the bench 308. It folds the beam path and also allows for alignment by adjustment of its kinematic mount 340K. Light from a fixation target display 350 and to alignment camera 354 are transmitted through the dichroic mirror 340. The fixation target is generally a two-dimensional display possessing adequate resolution to generate a visible image to which the patient will be instructed to direct their gaze.

A telescope lens group 352 locates the fixation target display 350 at infinity from the perspective of the patient's eye and its focus. The dichroic mirror 340 allows the green fixation target light to be transmitted to the patient and visible light from the patient to be transmitted to the alignment camera 354. Its advantage versus a long-pass dichroic is that the OCT beam is reflected instead of transmitted, which should avoid self-coherence and/or multireflection into the system. A 50/50 beamsplitter 356 couples light to the camera while transmitting light from the target to the patient.

In the sample arm, an achromat ocular lens 342 conditions the light so that the line is in focus on the retina, counteracting the eye's lens. The achromat ocular lens 342 is installed on a linear motion rail 341 and moved by a linear motion actuator 341A to adjust the lens position based on the patient's refractive error.

The light from the reference arm 320 and the sample arm 322 is combined in beamsplitter 318 and directed to the line scan or line field sensor 228. The linear array of the sensor 228 extends in the y-axis direction. A relay lens 360 is currently a triplet. This triplet is a Steinheil Triplet specifically, because it, with a single lens, provides a finite conjugate with relatively good aberration performance. A camera mirror 361 is mounted on a kinematic mount 361K to enable alignment of the interference beam to the line-scan camera 320.

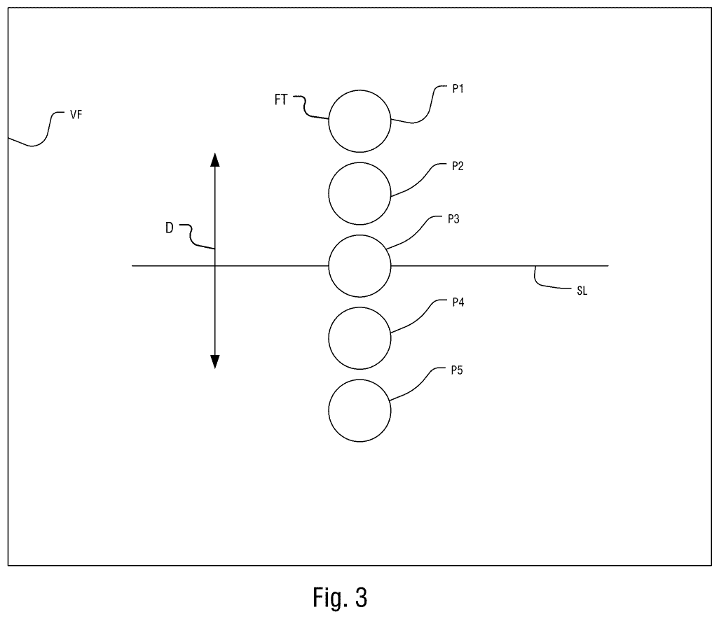

FIG. 3 shows the control of the fixation target FT generated by the fixation target display 350 relative to the scanline SL of the line-field non-scanning swept OCT system 200.

In more detail, a patient using the system 200 will see a fixation target FT in their visual field VF. Also, the patient will typically see the scanline SL generated by the system 200 and projected into the patient's eye. Whether the scanline is visible or not depends on the wavelength of operation of the laser 100. In the present example, at the power levels often used, the 840 nm laser emission will be visible to the patient.

In general, the SOM 230 controls the fixation target display 350 to move the fixation target FT in a direction that is orthogonal to the major axis of the scanline SL, see arrow D.

Preferably, the fixation target is sequentially moved from position P1 through positions P2, P3, P4, and P5. At each position, the OCT system 200 captures a separate B-scan. In this way, the central fovea of the patent is scanned without requiring a scanning capability for the system 200. Thus, the cost of the system can be reduced as a separate scanning galvanometer is no longer required.

In general, the number of B-scans can be any size. At least 3 are often necessary. Collecting 5 or 6 is usually sufficient for some patients to build up a C-scan. In other examples N B-scans are collected at N different locations, wherein N is 10 or more by moving the fixation target from position P1 through PN.

The distance between the successive B-scans as measured on the retina is usually greater than 20 μm. Preferably the retina is scanned over 1 millimeter extending to both sides of the fovea. In one example, 5 B-scans are collected at 250 μm steps, at −500 μm, −250 μm, 0 μm at fovea, +250 μm, and +500 μm.

In a preferred embodiment, this line is at least 5 millimeters (mm) long, but is preferably longer at 6 mm or more or 8 millimeters or more.

In operation, the laser 100 is swept in wavelength so that its frequency changes preferably linearly with time over the sweep or B-scan period. A linear frequency sweep is often desirable because it allows for efficient use of the camera sample rate and avoids the need to resample.

For the linear sweep in frequency, the angle of the filter 130 must be tuned in a non-linear fashion. As shown, the rate of change of the angle of the filter 130 slows with increasing angle and shorter wavelengths, higher frequency of the laser emission 102. This tuning curve for linear frequency sweeping is produced by and/or stored in the tuning curve module 166.

As the laser 100 begins its sweep, a trigger signal initiates the capturing of the line interference signals as the laser tunes. Preferably, at least 250 line interference signals are captured by the line-scan camera 228 within the sweep period of the laser 100. Currently more than 500 line interference signals, such as 1000 line interference signals or more are captured.

Preferably, the sweep period is less than 0.05 seconds and preferably less than 0.02 seconds. In the current examples, a sweep period of about 0.01 seconds or less provides acceptable B-scans of the human eye despite microsaccades and other movement.

Returning back to FIGS. 1 and 2, some examples further include a fixation target display for both the eye being examined and the patient's other eye.

Projecting a fixation target binocularly is useful where central fixation, in at least one eye, is preserved. Therefore, the eye with no central fixation can therefore still be examined by the system since the patient will be able to accurately follow the moving fixation target with either eye leveraging yoked eye movements.

In the FIG. 2, a second fixation target display 350′ is provided along with a second telescope lens group 352′ that locates the fixation target display 350′ at infinity for the non-examined eye. A separate ocular lens 342′ can also be provided. Both fixation target displays 350, 350′ are then controlled to move the fixation target together by the system on module 230.

In other examples, the single fixation target display is projected into both eyes using a beamsplitter.

While this invention has been particularly shown and described with references to preferred embodiments thereof, it will be understood by those skilled in the art that various changes in form and details may be made therein without departing from the scope of the invention encompassed by the appended claims.

What is claimed is:

1. A line-field optical coherence tomography (OCT) system, comprising:
a swept-source OCT module configured to project a fixed OCT scanline onto a retina;
a line-scan image sensor configured to detect interference light from the retina along the fixed OCT scanline;
a fixation target display positioned to project a fixation target into a field of view of an eye under examination; and
a system-on-module configured to:
control the fixation target display to sequentially present the fixation target at a plurality of positions orthogonal to the fixed OCT scanline,
trigger a wavelength sweep of a tunable laser to acquire a B-scan along the fixed OCT scanline at each fixation target position, and
compile the B-scans acquired at the plurality of fixation target positions into a composite image of a region of the retina,
wherein the composite image is acquired without mechanically scanning the OCT beam across the retina.

2. The system of claim 1, wherein the fixation target is sequentially presented at five positions corresponding to retinal locations spaced apart by about 250 micrometers or more.

3. The system of claim 1, wherein the fixation target positions cover a retinal region extending at least one millimeter on either side of the fovea.

4. The system of claim 1, wherein the plurality of fixation target positions comprises at least three positions.

5. The system of claim 1, wherein the fixation target display is further configured to present fixation targets to both eyes of the patient.

6. The system of claim 1, wherein the system is configured for home use by a patient for monitoring progression of diabetic retinopathy.

7. A method of generating a composite retinal image using a line-field optical coherence tomography (OCT) system, comprising:
positioning an eye so that a fixed OCT scanline and a fixation target are visible;
controlling a fixation target display to present the fixation target at a first position orthogonal to the fixed OCT scanline;
triggering a wavelength sweep of a tunable laser;
detecting interference light along the fixed OCT scanline with a line-scan image sensor to generate a first B-scan;
storing the first B-scan in memory of a system-on-module;
updating the fixation target display to present the fixation target at a second position orthogonal to the fixed OCT scanline;
repeating the triggering, detecting, and storing steps for each fixation target position until a predetermined number of positions has been completed; and
compiling the B-scans into a composite retinal image without mechanically scanning the OCT beam.

8. The method of claim 7, wherein the fixation target positions correspond to retinal locations spaced apart by about 250 micrometers or more.

9. The method of claim 7, wherein the fixation target positions cover a retinal region extending at least one millimeter on either side of the fovea.

10. The method of claim 7, wherein the plurality of fixation target positions comprises at least three positions.

11. The method of claim 7, further comprising projecting fixation targets to both eyes of the patient.

12. A non-transitory computer-readable medium storing instructions that, when executed by a processor of a system-on-module in a line-field optical coherence tomography (OCT) system, cause the system to:
control a fixation target display to present a fixation target at a plurality of positions orthogonal to a fixed OCT scanline projected onto a retina;
for each fixation target position, trigger a wavelength sweep of a tunable laser, acquire interference data from a line-scan image sensor along the fixed OCT scanline, and process the interference data into a B-scan; and
compile the B-scans into a composite retinal image without mechanical scanning of the OCT beam.

13. A binocular fixation optical coherence tomography (OCT) system comprising:
a swept-source OCT module configured to project a fixed scanline into one eye of a subject;
a binocular fixation target display configured to present a fixation stimulus visible to a second eye;
a detector configured to capture OCT interference data along the fixed scanline while the fixation stimulus is presented at different positions; and
a controller configured to combine OCT images from the different fixation stimulus positions into a composite image of the retina.
